(19) United States
(12) Patent Application Publication
(10) Pub. No.: US 2004/0096876 A1
(43) Pub. Date: May 20, 2004

(54) Quantitative analysis via isotopically differentiated derivatization

(76) Inventors: Steven J. Locke, Williamswood (CA); Devanand Pinto, Halifax (CA)

Correspondence Address: MORRISON & FOERSTER LLP, 755 PAGE MILL RD, PALO ALTO, CA, 94304-1018, US

(21) Appl. No.: 10/621,958

(22) Filed: Jul. 16, 2003

(30) Foreign Application Priority Data
Jul. 16, 2002 (CA) 2,393,726

(51) Int. Cl. C12Q 1/68; G01N 33/53

(52) U.S. Cl. 435/6; 435/7.1

(57) ABSTRACT

A method for the quantitative analysis of three or more samples containing molecules having an amine bearing an active hydrogen by mass spectrometry is disclosed. The method involves the chemical derivatization of amines in the sample by reacting the samples with isotopically labelled reagents, resulting in the isotopic labelling of the alklyamine derivatives. The method allows for the simultaneous examination and analysis of many samples. The method is particularly useful in the field of proteomics.

### FIELD OF THE INVENTION

The field of the invention is quantitative analysis coupled with mass spectrometry. In particular, the invention relates to the quantitative analysis of molecules such as proteins having an amine bearing an active hydrogen by mass spectrometry.

### BACKGROUND OF THE INVENTION

The emerging field of proteomics involves the characterization of the total protein repertoire of a biological sample. As there are numerous proteins in most samples, proteomics involves a high-throughput analysis and relies on the integration of significant advances recently achieved in two-dimensional (2D) gel electrophoretic separation of proteins, mass spectrometry and bioinformatics. As a result of these advances, it is now possible to obtain profiles of protein expression with much greater sensitivity and reproducibility. However, these analyses, though fast and easily automated, are not reliably quantitative.

Most present quantitative methods suffer from a major flaw, which may also affect qualitative comparisons, arising from lack of adequate reproducibility of 1-D and 2-D gel electrophoresis, or of other separation techniques for proteins such as immuno-affinity chromatography. It can be difficult to reliably replicate results obtained from two 2-D gel plates or chromatography columns. Therefore, it remains difficult to compare protein profiles from cells from two samples, for example in a normal healthy state with those in a diseased state.

Some recent advances in this science allow the protein extracts from two samples to be run on the same gel or column at the same time, thus avoiding all the ambiguities arising from variability in the separation procedure. However, these methods suffer from limitations in other ways.

For example, Oda et al., (1999) describe a method in which two cell cultures can be compared. The cells are cultured on growth media isotopically enriched by ¹⁵N-leucine. This method has the advantage that no chemical derivatization is required, and the two cultures to be compared can be mixed (and thus subjected to the identical conditions) before any of the subsequent analytical processing is done. However, it is applicable only to cases in which the appropriate cells can be grown in culture and is not applicable to larger organisms. Furthermore, it is limited to comparing two cultures.

Geng et al., (2000) describe a method using a lectin column to select glycoproteins from different proteomes which are subsequently labelled by acetylation of amino groups with N-acetoxysuccinimide or d₃-C¹—N-acetoxysuccinimide. However this method involves separate digestion of the two samples prior to labelling which can affect quantitation. In addition, the basic character of the peptide lysines are masked by the labelling reaction. This results in the positive ion spectra being dominated by Na⁺ and K⁺ adducts and sequencing by MS/MS is more difficult. Furthermore, the method disclosed is limited to comparing two samples.

Ji et al., (2000) describe a method using immobilized metal affinity chromatography after isotopic labelling to select for histidine-containing peptides. However, this method is limited to histidine residues, which are not very abundant (the abundance of histidine is estimated to be approximately 2%). In addition, this method can result in loss of post-translational modifications. Numerous proteins are regulated by post-translational modifications.

Mirgorordskaya et al., (2000) disclose a method involving a comparison of the MALDI intensity of a protein sample hydrolysed by chymotrypsin-like duodenase in ¹⁶O-water to a known concentration of the same sample that has been hydrolysed in ¹⁸O water to produce ¹⁸O-labelled internal standards. However, this technique is limited in that it requires separate digestion of the two samples which can affect quantitation. Another limitation of methods that use ¹⁸O water is the potential for exchange of other oxygen groups in the peptide after the initial reaction with trypsin. This results in additional peaks appearing for one peptide. As a result, the sensitivity decreases and the complexity of the spectrum increases which can affect the accuracy of the quantitation. Furthermore, the method disclosed is limited to comparing two samples. Yao et al., (2001) disclose a similar method employing trypsin.

Munchbach et al., (2001) describe a method of selective N-terminal acylation of peptides with 1-([d₀-or d₄]-nicotinoloxy) succinimide esters. The process involves initial blockage of the ε-amino of protein. lysines with succinic anhydride followed by enzymatic digestion with Asp (Glu) C protease. The peptides produced are selectively N-terminal acetylated with one of the isotopically labelled succinimide esters and then analysed by electrospray or MALDI. However, this technique alters the chemical properties of the lysine residues. The basicity of the lysine residue is a critical parameter for isoelectric focusing (the first step in 2D gel electrophoresis), therefore, any labelling process should aim to minimize the change in basicity between the native and labelled lysine. This method converts the lysine from a primary amine to an amide with an attached pyridine, thus greatly altering the basicity of lysine. This will have an effect on the isoelectric focusing of the protein. As a result of this drawback, labelling was done after 2D gel separation (each sample had to be run on a separate gel), which can affect the accuracy of the quantitation. Furthermore, the method disclosed is limited to comparing two samples.

Goodlett et al., (2001) disclose a method in which the protein mixtures from two different samples are enzymatically hydrolysed with trypsin, followed by the labelling of the resultant peptides with either d₀ or d₃-methanol. However, this method labels the peptides after digestion, and although the basic character of the lysine is retained, the acidic nature of the aspartic/glutamic acid is lost. Furthermore, the method disclosed is limited to comparing two samples.

Smith et al., (2001) employ high performance Fourier transform-ion cyclotron resonance (FTICR) mass spectrometry to generate accurate mass tags for peptides or intact proteins. The principle is that the accuracy of the mass measurement will be such that the mass of the peptide is unique amongst all the other peptide masses predicted for a genome. However, this method relies on the high mass accuracy attainable with the FTICR mass spectrometer, which is very costly.

Three recent papers describe methods of quantitative proteomics via cysteine-residue labelling.

Gygi et al., (1999) describe a method of chemical derivatization in which cysteine residues are targets. This approach involves differential labelling of the native proteins immediately after extraction of the protein complement from the samples, followed by mixing of the samples to be compared before any further steps are performed. However, the particular derivatization reaction employed involves chemical attachment of biotin groups to cysteine residues in the proteins, which can result in loss of potentially important post-translational modifications, particularly phosphorylation. Further, cysteine residues are relatively rare in protein samples. This method was developed specifically for use with affinity purification rather than 1-D or 2-D gel electrophoresis, and one can foresee problems in its application to these important separation techniques. Furthermore, this method allows only two samples to be compared.

Wang et al., (2001) disclose a method involving the N-acylation of peptides with d₀ or d₄-succininic anhydride following the separate tryptic digestion of protein samples. Cysteine-containing peptides are selected by covalent chromatography based on thiol-disulfide exchange with a thiopropyl Sepharose™ gel. However, this method suffers from the same limitations as Gygi et al. (1999) and only two samples can be compared.

Conrads et al., (2001) use ¹⁴N/¹⁵N metabolic labelling but combines this with biotin labelling of cysteine residues, in a similar manner to Gygi et al., (1999). Again, the method suffers from the same limitations as Gygi et al., (1999) and only two samples can be compared.

Three recent papers describe methods of protein quantification using phosphoprotein-selective reagents and methods.

For example, Weckwerth et al., (2000) present a method for the relative quantitation of phosphoproteins to compare the phosphorylation status of proteins under two different states. The method involves chemical treatment of the proteins to selectively remove phosphate from phospho-Ser/Thr followed by addition of ethanethiol or ethane-d₅-thiol. However, the isotopically-specific chemical derivatization occurs at a late stage in the analysis (following proteolysis of the proteins). In addition, there are too many steps in which the two protein extracts are processed separately, and then made to react with chemical and biochemical reagents in different reaction mixtures whose reproducibility cannot be guaranteed. Thus, the relative abundances of target proteins derived from such procedures are liable to systematic errors of unknown magnitude in any given case. Furthermore, the method disclosed is limited to comparing two samples.

Molloy et al., (2001) describes a similar method to Goodlett et al., (2001), except that a mixture of two different non-isotopically labelled alkanethiols is used for the qualitative detection of phosphopeptides. However, this method is selective for phosphoproteins. Furthermore, the method disclosed is limited to comparing two samples.

Finally, Goshe et al., (2001) presents a method combining stable isotope and biotin labelling to enrich and quantitatively measure differences in the O-phosphorylation states of proteins. However, this method is selective for phosphoproteins and only two samples can be compared.

Thus, although there have been some recent attempts at quantitative proteomics, these approaches are limited in that (1) if the proteins are tagged or labelled, the methods are limited to comparing only two samples, (2) the approaches are too cumbersome and problematic, (3) the approaches involve labelling cysteine residues, which are generally rare in protein samples, and which can result in loss of post translational modifications, (4) they are limited to affinity purification, (5) they are limited to samples that can be cultured in vitro, (6) they require use of a costly mass spectrometer and (7) they are limited to detection of phosphoproteins.

Thus, there is a need to develop an effective method of quantitative proteomics which allows for any one or more of: (1) simple and easy labelling of the sample, (2) comparison of more than two samples simultaneously, (3) simultaneous preparation of two or more samples to avoid variability between samples, (4) labelling residues that are common and not involved in post-translational modification, (5) use of any method of protein separation, including 1-D and 2-D gel electrophoresis, (6) analysis of any sample, and is not limited to samples that can be cultured in vitro, and (7) use of an inexpensive mass spectrometer (MS).

### SUMMARY OF THE INVENTION

According to one aspect of the invention, there is provided a method for the simultaneous quantitative analysis of at least three samples of molecules, comprising (i) reacting the molecules of each sample with a set of at least two isotopically labelled reagents, wherein each set of isotopically labelled reagents is differentially labelled, resulting in at least three differentially and isotopically labelled derivatives of molecules, (ii) combining the derivatized molecules in a preparation for examination by mass spectrometry, and (iii) examining the preparation by mass spectrometry.

According to another aspect of the invention, there is provided a method for the quantitative analysis of a sample of molecules having an amine bearing an active hydrogen, comprising (i) reacting the molecules, with isotopically labelled reagents resulting in the reductive alkylation of the amines to their alkylamine derivatives, such that the alkylamine derivatives are isotopically labelled in a preparation for examination by mass spectrometry and, (ii) examining the preparation by mass spectrometry.

According to another aspect of the invention, there is provided a method for the quantitative analysis of two or more samples of molecules having an amine bearing an active hydrogen, comprising (i) reacting the molecules in each sample with isotopically labelled reagents resulting in the reductive alkylation of the amines to their alkylamine derivatives, such that the alkylamine derivatives are isotopically labelled, (ii) combining the derivatized molecules in a preparation for examination by mass spectrometry, and (iii) examining the preparation by mass spectrometry.

The above-described methods may comprise an additional step of cleaving the derivatized molecules in the preparation into fragments, prior to examining the preparation by mass spectrometry and an additional step of denaturing the molecules prior to reacting the molecules with isotopically labelled reagents.

The step of examining the preparation by mass spectrometry may comprise introducing the preparation containing the derivatized molecules or fragments to a mass spectrometer using electrospray ionisation, where the electrospray ionisation is selected from a group consisting of nanospray, pneumatically assisted electrospray, ionspray and turboionspray.

The above-described methods may comprise an additional step of separating the derivatized molecules in the preparation before examining the preparation by mass spectrometry. The step of separating the derivatized molecules may use a separator selected from a group consisting of 1-D gel electrophoresis, SDS-PAGE, isoelectric focusing, 2-D gel electrophoresis, zone electrophoresis, isotachophoresis, ion exchange chromatography, normal phase chromatography, reverse phase chromatography, hydrophobic interaction chromatography, size exclusion chromatography and any combination of these separators.

The above-described methods may further comprise an additional step of separating the fragments after cleaving the derivatized molecules in the preparation. The step of separating the fragments may use a separator selected from a group consisting of liquid chromatography, high performance liquid chromatography and capillary electrophoresis.

The above-described methods may further comprise an additional step of analyzing the preparation after examining the preparation by mass spectrometry. The step of analyzing the preparation may be selected from a group consisting of collision-induced dissociation in a mass spectrometer operating in MS/MS mode, peptide mass fingerprinting, peptide mapping, Edman sequencing and sequencing by sequential amino acid cleavage.

The above-described methods may further comprise an additional step, after the step of analyzing the preparation of sequencing the molecule.

The isotopically labelled reagents may be an aldehyde and a reducing agent. The aldehyde may be formaldehyde and acetaldehyde and the reducing agent may be sodium cyanoborohydride, sodium borohydride, dialkyl borane complexes and pyridine borane complexes.

The sample may include cells, cellular extracts, sub-cellular extracts, cellular lysates, peptides, proteins, drugs, toxins, antibodies and pollutants.

The above-described methods may use any mass spectrometer, for example: (i) Fourier transform—Ion cyclotron resonance mass spectrometers (FT-ICR-MS), (ii) Time of Flight mass spectrometers (TOF-MS, TOF-TOF-MS), (iii) Ion trap mass spectrometers (IT), (iv) Quadrupole mass spectrometers (Q-MS and QqQ-MS), (v) Ion mobility mass spectrometers (IM-MS), Quadrupole (or hexapole, octapole)—Time of Flight mass spectrometers (Q-TOF, and Qq-TOF), and (vii) Ion trap—Time of flight mass spectrometers (IT-TOF). In addition, the mass spectrometer may be combined with an ionisation source, for example, electrospray ionisation, matrix-assisted laser desorption and ionisation (MALDI), field desorption, thermal desorption and laser desorption.

Another aspect of the invention, is to provide a preparation of three samples of molecules for simultaneous quantitative analysis by mass spectrometry, each sample comprising differentially and isotopically labelled derivatives of molecules, each sample resulting from a reaction of a set of at least two isotopically labelled reagents with the molecules.

Another aspect of the invention is to provide a preparation of a sample of molecules comprising isotopically labelled derivatives resulting from the reductive alkylation of the amines to alkylamine derivatives by isotopically labelled reagents.

Another aspect of the invention, is to provide a preparation of two or more samples of molecules for the simultaneous analysis by mass spectrometry, each sample comprising differentially and isotopically labelled derivatives of molecules resulting from the reductive alkylation of the amines to alkylamine derivatives by isotopically labelled reagents.

Another aspect of the invention is to provide the use of a mass spectrometer for the analysis of a sample according to any of the above-described methods.

Another aspect of the invention is to provide a kit comprising isotopically labelled reagents and instructions to follow the methods of quantitative analysis of any of the above-described methods.

Another aspect of the invention, is to provide a method for the quantitative analysis of two or more cellular extracts comprising molecules having an amine bearing an active hydrogen, comprising (i) reacting the molecules of the extracts with isotopically labelled reagents resulting in the reductive alkylation of the amines to their alkylamine derivatives, such that the alkylamine derivatives are isotopically labelled, (ii) combining the derivatized molecules of the extracts in a preparation, (iii) separating the molecules, (iv) enzymatically cleaving the molecules into fragments, (v) separating the fragments, (vi) examining the fragments by mass spectrometry, and (vii) sequencing the fragments.

### BRIEF DESCRIPTION OF THE FIGURES

FIG. 1 is a MS spectrum of the same protein from two samples, both samples were labelled with formaldehyde, the first sample was reduced with sodium cyanoborohydride and the other sample was reduced with sodium cyanoborodeuteride, the samples were combined and digested with chymotrypsin and analysed using ESI-MS.

FIG. 2 is a MS spectrum as in FIG. 1, except that three samples were labelled. In addition the peptides were triply charged.

FIG. 3 is a LC-MS spectrum demonstrating that the tags are compatible with gel electrophoresis and also demonstrating the added specificity obtained through labelling. The preparation consisted of two BSA samples, both samples were labelled with formaldehyde, the first sample was reduced with sodium cyanoborohydride and the other sample was reduced with sodium cyanoborodeuteride, the samples were combined and the preparation was analysed by SDS-PAGE gel electrophoresis, excised, digested with chymotrypsin and analysed by LC-MS. The peptides indicated by arrows contain the label and can be distinguished from peptides arising from contaminants (such as trypsin or keratin) and unlabelled peptides based on their isotopic pattern.

FIG. 4 is a tandem MS spectrum of two peptides with isotopic labelling. The first sample was labelled with formaldehyde and sodium cyanoborohydride. The second sample was labelled with formaldehyde and sodium cyanoborodeuteride. This data was obtained during the same LC-MS analysis described in FIG. 3.

FIG. 5 is a composite of 4 spectrums. Top left, nanoelectrospray spectrum; bottom left, total-ion chromatogram for the m/z range of 475-485; top right, MS spectrum for the first peak consisting of a triplet of doubly charged peptides; bottom right, MS spectrum for a second peak consisting of a triplet of triply charged peptides.

FIG. 6 is a MS spectrum of two samples of BSA differentially labelled, combined, digested using trypsin and analysed by LC-MS. Trace A shows the MS spectrum of a pair of labelled tryptic peptides and Trace B shows the extracted ion chromatograms for the same two peptides.

FIG. 7 is a 2D gel separation of membrane proteins from Aeromonas salmonicida. Panel A is a separation of proteins from one sample. Panel B is a separation of proteins from two samples grown under different conditions.

FIG. 8 is a composite of the molecular structures of various amines differentially labelled and analysed by MS and a mass spectrum of one of the analysed amines, 3-aminothiophenol.

FIG. 9 is a MS spectrum of 4 samples of amine containing molecules differentially labelled and analyzed by MALDI-MS.

FIG. 10 contains 2 MS spectra from the analysis of a pentalabelled ovalbumin preparation. The preparation contains a mixture of ovalbumin labelled with five unique tags. The samples were each labelled with a unique tag, combined, run on an SDS-PAGE gel, digested with trypsin and analysed by MALDI-QqTOF-MS. 500 fmol of protein was loaded on the MALDI target. The protein ratios were 3:1:3:1:3 (top trace) and 1:3:1:3:1 (bottom trace). Note that a mixture of labelled and unlabelled peaks are present, both of which can be used for identification.

FIG. 11, is an expansion of FIG. 10 in the region of the labelled peptides.

FIG. 12 contains the Tandem MS spectra of peaks with m/z ratios of 1072.8 (middle trace), 1080.8 (bottom trace) and 1088.8 (top trace). The spectra were acquired individually using MALDI-QqTOF-MS/MS. Note that the fragmentation pattern is identical for all of the labels. In addition, the low-mass ions at 129, 133 and 137 are indicative of the specific label present.

FIG. 13 is the MS spectrum obtained from a protein isolated from Candida albicans. This fungus was grown under conditions favouring the yeast form (30° C.) and conditions favouring the hyphal form (37° C.+serum). Proteins were extracted, isotopically labelled, combined, separated by SDS-PAGE electrophoresis, digested with trypsin and analysed by MALDI-QqTOF-MS.

FIG. 14 contains the Tandem MS spectra of peaks with m/z ratios of 1096.7 (top trace) and 1102.8 (bottom trace) as in FIG. 13. The spectra were acquired individually using MALDI-QqTOF-MS/MS. Note that the fragmentation pattern is identical for both of the labels. Both spectra matched to the protein enolase.

### DEFINITIONS

Active hydrogen means a hydrogen directly bonded to a nitrogen atom.

CID means collision induced dissociation.

Electrospray means a source of ionization in which a liquid sample is nebulized from a tube due to an applied potential, in the presence or absence of a nebulizing gas, which gas, if present, also provides a charge to the droplet, and in which the resultant charged droplet evaporates and fragments yielding small charged droplets or charged molecular ions.

ESI-MS means electrospray ionization mass spectrometer.

FTICR means Fourier transform—Ion cyclotron resonance mass spectrometer.

HPLC-MS means high performance liquid chromatography coupled with a mass spectrometer.

IM-MS means ion mobility mass spectrometer.

IT-MS means ion trap mass spectrometer.

Laser Desorption Ionization means a source of ionization in which ions are produced from a sample adsorbed onto a surface, such as silicon, by exposure to laser irradiation, typically at low pressure (although recently atmospheric pressure is being used).

MALDI means a source of ionization (Matrix Assisted Laser Desorption Ionization) in which ions are produced from a sample mixed with a matrix (typically analysed in crystalline form) by exposure to laser irradiation, typically at low pressure (although recently atmospheric pressure is being used).

MS/MS means tandem mass spectrometry, in which ions are selected, caused to fragment and the fragments analysed. It can therefore be used for structural and sequencing studies.

m/z means the mass to charge ratio.

QMS or QqMS means quadrupole mass spectrometer.

QTOF-MS means quadrupole time of flight mass spectrometer.

Sample means any matter or composition containing or suspected of containing a substance being identified or measured in an analysis. The sample may contain molecules having an amine bearing an active hydrogen.

Sequencing means obtaining the sequence of a protein or protein fragment through correlation of a measured value with values derived from a database or directly through fragmentation in a mass spectrometer or by chemical cleavage methods.

ToF-MS means Time of flight mass spectrometer.

### DETAILED DESCRIPTION OF THE INVENTION

The invention is an approach to quantitative analysis of any sample based on chemical derivatization with a well-characterized and selective reactivity to achieve the introduction of an isotopically labelled tag. The invention is particularly useful for proteome analysis. The method involves the reductive alkylation of amine groups to their alkylamine derivatives by the action of an added aldehyde (for example formaldehyde) and a reducing agent, optionally a Schiff base reducing agent, for example sodium cyanoborohydride. This occurs mostly on the side chains of the amino acid lysine, but can also occur on the N-terminal amino group of the protein. The labelled tags are electrophoretically and chromatographically similar but have a sufficient mass shift to be able to easily differentiate them by a mass spectrometer.

There are many benefits to this invention. Among them are the following:

The method is applicable to virtually any sample of molecules having an amine bearing an active hydrogen. The most common use envisioned is for protein samples, and in particular use in the field of proteomics. The amino acid lysine is present in almost all proteins. Typically, lysine comprises 3-8% of the total amino acid content of proteins. For example, bovine serum albumin (BSA, about 67 kDa), contains 60 lysine residues per molecule. The reductive alkylation and enzymatic cleavage generates a specific pattern of labelled peptides for quantitation. The specific patterns produced greatly simplifies the analysis of the samples by mass spectrometry. The presence of multiple representative peptides from each protein provides a high degree of quantitative accuracy and increases the ability of measuring low-abundance proteins.

More than two samples can be analysed simultaneously. The method can provide a minimum of eight distinct labels, thus enabling eight different samples, such as eight extracts, to be probed simultaneously. This can be accomplished by differentially labelling two reagents, using both hydrogen and carbon isotopes (for example, as shown in Table 1), which results in eight different masses of the chemically derivatized products. Existing methods allow only two states to be probed at a time. This is because most reagents used for protein quantitation consist of a light tag (natural abundance isotopes) and heavy tag (for example, fully deuterated) since synthesis of tags that are partially deuterated is difficult. In addition, due to the low throughput of current proteomic technologies, many labs are finding the comparison of more than two samples unmanageable. The present method can use a set of at least two differentially isotopically labelled reagents. This combination enables the number of samples to be doubled, i.e. each aldehyde can be used with one of two different reducing agents. Therefore, it is possible to expand the number of labels even further by using radioactive formaldehyde and/or cyanoborohydride.

The present invention permits isotope-labelled chemical derivatization of an entire protein extract, in a way, which is generally applicable, and not intrinsically limited to cultured cells. The entire extracts to be compared are derivatized with reagents which are chemically identical but have different stable isotope contents (typically deuterium). This can be done prior to mixing of these different derivatized extracts, separation of the constituent proteins (now mixtures because of the different labelling), proteolytic digestion and analysis by mass spectrometry.

The technique allows for the simultaneous separation and detection of the samples containing the isotopic tags. The basis of the new advance is to run the protein extracts from two (or more) samples on the same gel or column at the same time, thus avoiding all inaccuracies arising from variability in the separation procedures. The method mixes together the protein extracts to be compared after the protein extraction and chemical derivatization steps. Both of these steps are essentially 100% efficient so that no distortion of relative abundance can arise.

The invention involves derivatization of lysine residues, which are fairly abundant in nature. Lysine residues are generally accessible to chemical reagents as they are less likely to be buried in the hydrophobic core of a protein in aqueous solution. In addition, lysine residues are more likely to be in a reactive form unlike cysteines which typically exist in a disulphide form that must be reduced prior to derivatization.

As a result of the multiple-labelling capability, the invention can distinguish among multiple samples in a single experiment. Also, the choice of lysine yields more labelled proteolytic peptides per protein molecule, with a consequent increase in sensitivity and accuracy of quantitation.

The chemistry of the derivatization is established and essentially quantitative under the conditions established in the invention. For formaldehyde and sodium cyanoborohydride, each lysine is labelled with two methyl groups under mild reaction conditions. No other amino acids can react with these reagents under the conditions employed, other than omithine, which is one of the very uncommon amino acids with a side chain group chemically similar to that of lysine.

The molecular mass and isoelectric point of the labelled proteins are not significantly different from those of the unlabelled protein. For example, for BSA (bovine serum albumin), labelling of all lysines with formaldehyde results in a 2.5% increase in the molecular weight. This difference in both molecular weight and pI becomes even less significant for proteins labelled with different isotopes of the same reagent. Thus, the differentially labelled forms of a particular protein from a number of samples co-electrophorese, meaning the different forms of a protein are indistinguishable by 1D or 2D gel electrophoresis. In addition, the differentially labelled samples have high chromatographic similarity. Due to the nature of the labels developed, the chromatographic behaviour of the differentially labelled peptides is very similar. Therefore, the labelled peptides co-elute during liquid chromatography (LC) separation. This is important for accurate quantitation of the samples, since peptide intensity in LC-MS analysis is dependant on sample conditions at the time of elution. In other words, relative quantitation requires that the peptides co-elute.

The presence of a multiplet of peaks of predetermined spacing in the mass spectrum is easily detected and characteristic of peptides from the protein in different samples. The relative intensities of the signals would provide quantitation of the relative amounts of the protein in the different samples.

The technique is applicable to any sample containing a molecule having an amine bearing an active hydrogen, including non-protein molecules. For example, likely candidates include, but are not limited to cells, cellular extracts, sub-cellular extracts, cellular lysates, peptides, proteins, drugs, antibodies, pollutants and toxins.

The method can be used in a manner that does not affect post-translational modifications. The detection of post-translational modifications is important because numerous vital activities of proteins are regulated by post-translational modifications. For example, cell cycle progression, cellular differentiation, development, hormone response and protein kinase activation are all modulated by post-translational modifications.

The method can employ a wide range of separation techniques. Any separation technique can be used. For example, the method can employ 1-D gel electrophoresis, 2-D gel electrophoresis, zone electrophoresis, isotachophoresis, ion exchange chromatography, normal phase chromatography, reverse phase chromatography, hydrophobic interaction chromatography, size exclusion chromatography and combinations thereof.

In addition, the label does not interfere with the ability to perform sequencing experiments through collision-induced fragmentation (CID). The CID process follows a similar pattern to that observed with non-labelled peptides.

### Methods and Materials

Any sample of molecules can be quantified by the present invention. Molecules having an amine bearing an active hydrogen can be quantified by the present invention. The most common samples envisioned are protein and peptide samples from cells, cellular extracts, sub-cellular extracts and cellular lysates. Samples may also include other peptides, proteins and drugs, antibodies, pollutants, and toxins.

The following methods will be described using protein samples as examples, but it is understood that any sample comprising molecules having an amine bearing an active hydrogen may be used.

### 1) Labelling, by Reacting the Molecules in the Sample with Isotopically Labelled Reagents

The method employs the reductive alkylation of amine groups to their alkylamine derivatives. Reductive alkylation is a convenient method for converting amino groups in proteins and peptides to their alkylamine derivatives through the reduction of the Schiff base that forms between the ε-amino group of lysine residues and an added aldehyde. In the case of formaldehyde (CH₂O) and sodium cyanoborohydride (NaCNBH₃) as a reducing agent for the Schiff base, a primary amino group (mostly on sidechains of the amino acid lysine, but also the N-terminal amino group of the protein if this is not blocked) can be selectively and quantitatively converted to its dimethyl derivatives at slightly alkaline pH under very mild reaction conditions. The changes in the physical and chemical properties of the protein are minimal because of the small size of the introduced methyl groups (and therefore minimal changes in molecular weight). The charge of the amino group is usually also retained with a small alteration in the value of the isoelectric point, pI, so that the derivatized proteins will migrate in the two dimensions (molecular weight and pI) of 2-D gel electrophoresis almost exactly the same as the underivatized proteins.

In the case of a protein sample, the lysine residues can be derivatized with an aldehyde (for example formaldehyde, CH₂O) and a reducing agent (for example sodiumcyanoborohydride, NaCNBH₃), in the presence of an alkaline buffer (˜pH 8). The lysine residues representing a second sample can be derivatized with an isotopically labelled aldehyde (for example CD₂O), and the same reducing agent. Lysine residues representing other samples are derivatized with other isotopically labelled aldehydes (or acetaldehydes) and a reducing agent (NaCNBH₃). The number of samples that can be labelled can be further increased by the use of isotopically labelled reducing agents (for example, NaCNBD₃) instead of NaCNBH₃ to introduce different degrees of deuterium labelling. The tags are thus chromatographically similar but with a large enough mass shift to be able to be easily distinguished by a mass spectrometer.

The specific, optimal pH conditions for reductive alkylation are determined by the properties of the analyte. For example, in the case of proteins, conditions must be chosen to minimise cross-linking reactions. Specifically, the reducing agent should be added first and at a pH <7. This also minimises the formation of N-cyanomethyl adducts from impurities in the NaCNBH₃. However, too low a pH reduces the reaction efficiency since the pKa of ε-amino groups is ˜11. At pH 6.5, alkylation of the α-amino is favoured. Similarly, when NaCNBH₃ is used, reaction efficiency decreases above pH 8. Therefore, for complete methylation of proteins, a pH of 7-8 is recommended.

For other amino compounds, reductive alkylation is typically performed in organic solvents in the presence of 10-30% acetic acid. These amines are generally weaker bases than amines in proteins and, therefore, do not lose reactivity at low pH. In such cases, the acid is beneficial in that it accelerates the dehydration after reaction between the aldehyde and the amine.

Any aldehyde can be used. Typically, formaldehyde or acetaldehyde is used. Other aldehydes can be used, however, those that do not react quantitatively are not ideal for protein analysis. Other aldehydes may be suitable for small molecules where more extreme reaction conditions can be used to promote a quantitative reaction. Although, larger aldehydes tend to be less reactive due to steric hindrance.

Schiff base reducing agents that can be used include sodium cyanoborohydride and sodium borohydride. However, sodium borohydride is very reactive, and can further reduce the aldehyde to an alcohol prior to its reaction with the amine nitrogen. Other reducing agents can be used. For example, alkylborane or pyridine borane complexes can also be used for the reduction.

Optionally, a denaturing agent can be added to unfold the protein as is known to those skilled in the art in order to label buried lysine residues.

The reactants can be added in any order under almost any conditions. Preferably, however, the aldehyde is combined with the buffer and then mixed with the sample in the presence of the reducing agent. As is known to those skilled in the art, some care must be taken in using reaction conditions that prevent unwanted side-reactions such as crosslinking of the protein by the aldehyde.

As explained above, many different samples can be compared using different isotopically labelled reagents. Isotopic labelling techniques are known to those skilled in the art. For example, Table 1 lists differentially labelled formaldehyde and sodium cyanoborohydride reagents that can be used to differentiate samples.

TABLE 1
Differentially labelled formaldehyde and sodium cyanoborohydride.

| | Reducing | | Added Mass | |
|---|---|---|---|---|
| Formaldehyde | Agent | Solvent | per NH₂ | Δ Mass per NH₂ |
| CH₂O | NaCNBH₃ | H₂O | 28.0316 | 0.0000 |
| CH₂O | NaCNBD₃ | D₂O | 30.0474 | 2.0158 |
| ¹³CH₂O | NaCNBH₃ | H₂O | 30.0316 | 2.0000 |
| ¹³CH₂O | NaCNBD₃ | D₂O | 32.0474 | 4.0158 |
| CD₂O | NaCNBH₃ | H₂O | 32.0632 | 4.0316 |
| CD₂O | NaCNBD₃ | D₂O | 34.0790 | 6.0474 |
| ¹³CD₂O | NaCNBH₃ | H₂O | 34.0632 | 6.0316 |
| ¹³CD₂O | NaCNBD₃ | D₂O | 36.0790 | 8.0474 |

Although the preferred method of labelling amine groups is described above, the invention encompasses the use of other classes of amine reactive reagents. For example:

a) Isocyanates: Isotopic reagents consisting of isocyanates are very reactive toward amino groups. They may not be as stable as the aldehyde and Schiff base reaction described above, and may decompose during storage.

b) Isothiocyanates: Isotopic reagents consisting of isothiocyanates can also be used. They are generally less reactive than aldehydes and may hydrolyse in aqueous solution.

c) Succinimidyl esters: Isotopic reagents consisting of succinimidyl esters may also be used, although they are less reactive than aldehydes and tend to hydrolyse in aqueous solutions, especially at alkaline pH (>pH 9). Further, reactivity can be low for reagents that are not highly soluble in aqueous solution.

d) Sulfonyl halides: Isotopic reagents consisting of sulfonyl halides can be used but are generally quite unstable in aqueous solution, especially at pH values necessary for reactivity of amino groups.

e) Other aldehydes and ketones: Isotopic reagents consisting of higher aldehydes and ketones are possible but they may generally provide lower reactivity due to poor solubility and/or steric hindrance at the reactive carbonyl group.

In addition, other groups (other than amines) can be targeted on molecules to be analyzed, for example proteins. For example:

a) Thiol-disulfide: In proteins, thiol groups are present in cysteine residues. Generally, thiols exist in the oxidized cysteine form and must be reduced to the free thiol for reactivity. The thiol-reactive reagents generally fall into one of the following classes, iodoacetamides, maleimides, benzylic or aliphatic halides. Iodoacetamides are generally quite reactive toward thiols and form stable products but the reagents may be unstable in light, especially in solution. Maleimides are generally selective reagents for thiol modification but they can suffer from poor solubility and significant hydrolysis to the corresponding maleimic acid, especially above pH 8.

b) Alcohols: In proteins, alcohol groups are present in the form of serine, threonine and tyrosine residues as well as carbohydrate residues of glycoproteins. However, few reagents are selective for the hydroxyl group in aqueous solution, especially in the presence of the more nucleophilic amino and thiol groups. Furthermore, any reagent that would be reactive toward hydroxyl groups may be readily hydrolysed in aqueous solution.

c) Carboxylic acids: In proteins, carboxylic acids are present in aspartic and glutamic acid residues. These residues can be coupled to hydrazines and amines in aqueous solution with the aid of water-soluble carbodiimides as activating agents. In order to minimize side reactions such as intra- and inter-protein coupling via lysine residues, these reactions must be conducted in concentrated protein solutions and at low pH, using a large excess of the reagent.

### 2) Combining the Derivatized Molecules in a Preparation and Optional Separation

If more than one sample is being analyzed, the different protein samples can be combined. Optionally, for complex samples, the proteins are separated by methods known in the art, including 1D or 2D gel electrophoresis. Other methods for protein separation are known to those skilled in the art and include, but are not limited to: various modes of chromatography, including, ion-exchange, normal phase, reverse phase, hydrophobic interaction, size exclusion, various modes of electrophoresis, including zone, gel and isotachophoresis as well as combinations of any of the above methods. After separation of the proteins, the gel is stained by one of several established methods known in the art, for example silver, Coomassie Blue or metal based reverse staining. Since the different protein samples have been labelled with the same reagent, the behaviour during electrophoresis of a particular protein should be identical regardless of the isotopic label employed. Upon staining of the gel with a reagent such as silver nitrate, each band/spot contains a particular protein from each of the samples. The stained protein bands/spots are then excised.

### 3) Cleavage of the Derivatized Molecules to Yield Fragments

Optionally, samples can be cleaved to generate peptide fragments from the protein in the different samples. Although the conversion of the ε-amino group to a dimethylamino group renders the lysine residues unreactive to trypsin, the enzyme that is most commonly used to generate peptides for mass spectrometric analysis, trypsin will cleave a derivatized protein at the arginine residues (whose abundance in proteins is similar to that of lysine). Therefore, it is possible to perform tryptic digestion of labelled proteins and obtain accurate quantitative results using the disclosed method. Further, the proteins can also be cleaved by other enzymes such as chymotrypsin or by chemical means. The collision induced dissociation (CID) fragmentation of peptides generated by these methods is as informative as the fragmentation pattern of unlabelled tryptic peptides. Computer databases exist for sequencing of proteins from the chymotryptic peptides.

### 4) Separation of Fragments and Introduction into Mass Spectrometer

The peptide fragments can be introduced into the mass spectrometer, preferably using peptide separation methods known to those skilled in the art, including but not limited to liquid chromatography or capillary electrophoresis. Preferably, the method is High Performance Liquid Chromatography (HPLC). The sample(s) or peptide fragments can also be introduced without separation using methods known to those skilled in the art, including but not limited to, electrospray ionization methods such as nanospray, pneumatically assisted electrospray, ionspray and turboionspray.

### 5) Examining the Preparation Containing Derivatized Molecules or Fragments by Mass Spectrometry

The samples or fragments are then examined by mass spectrometry. In this step both the quantity and sequence identity of the proteins from the gel spot can be determined. Peptides from the different samples are quantified by measuring the relative signal intensities for the ions of identical sequence that are tagged with different isotopic forms of the reagents. For example, the peptides labelled with CD₂O and NaCNBH₃ prior to separation-digestion would have a molecular mass 4 Da higher than peptides from the same protein that were labelled with CH₂O and NaCNBH₃. For some commonly used enzymes in the cleavage step, the peptides show up in the mass spectrum predominantly as doubly-charged ions so the mass to charge spacing is half of the molecular mass spacing. The appearance in the mass spectrum of a doublet separated by 4 Da (molecular mass) is indicative of the protein from the two different samples. If a third sample is labelled with ¹³CH₂O and NaCNBH₃ and run on the same gel, a triplet in the mass spectrum with a separation of 2 Da occurs.

Many different mass spectrometers can be used in this invention as is known to those skilled in the art. They include but are not limited to:

(i) Fourier transform—Ion cyclotron resonance mass spectrometers (FT-ICR-MS)

(ii) Time of Flight mass spectrometers (TOF-MS, TOF-TOF-MS)

(iii) Ion trap mass spectrometers (IT)

(iv) Quadrupole mass spectrometers (Q-MS and QqQ-MS)

(v) Ion mobility mass spectrometers (IM-MS)

Hybrid-geometry mass spectrometers can also be used for the invention, examples include:

(vi) Quadrupole (or hexapole, octapole)-Time of Flight mass spectrometers (Q-TOF, and Qq-TOF)

(vii) Ion trap—Time of flight mass spectrometers (IT-TOF)

The above mass spectrometers can be combined with ionization sources as is known to those skilled in the art, which include but are not limited to, electrospray ionization at any flow rate, matrix-assisted laser desorption and ionization (MALDI), field desorption, thermal desorption and laser desorption from suitable substrates (i.e. titanium dioxide and silicon dioxide).

### 6) Analyzing the Derivatized Molecules or Fragments and Sequencing the Molecules or Fragments

Sequencing is an optional step. Sequencing information is generated by selecting peptide ions of a particular mass-to-charge ratio for collision-induced dissociation (CID) in the mass spectrometer operating in the MS/MS mode. Using sophisticated computer-searching algorithms, the resulting CID spectra is automatically correlated with sequence databases to identify the protein from which the sequenced peptide originated. Other modes of analysis as is known to those skilled in the art, include but are not limited to, peptide mass fingerprinting, peptide mapping, Edman sequencing and sequencing by sequential amino acid cleavage.

### 7) Kits

The present invention encompasses kits comprising isotopically labelled reagents and instructions to follow the methods of quantitatively analyzing a sample described herein. For example, the kits may include reagents for comparing at least two samples, comprising at least two sets of two differentially labelled reagents.

For example, kits for comparing two samples of amine containing molecules by the derivatization of molecules having an amine bearing an active hydrogen by the reductive alkylation of the amines to their alklyamine derivatives, may comprise one reducing agent (for example NaCNBH₃) and two differentially labelled aldehydes (for example, CH₂O and ¹³CH₂O). Alternatively, the kits may comprise two differentially labelled reducing agents (for example, NaCNBH₃ and NaCNBD₃) and one aldehyde (for example, CH₂O). Several combinations are available as is known to those skilled in the art, and can be seen, for example, in Table 1. The kits may also include differentially labelled reagents for comparing more than two samples. For example, a kit for comparing three samples, can include one reducing agent (for example, NaCNBH₃) and three differentially labelled aldehydes (for example, CH₂O, ¹³CH₂O and CD₂O). Accordingly, the kits may comprise many combinations of labelled reagents as required to differentiate two of more samples as is known by those skilled in the art.

The kits may also include software for the method of quantitatively analyzing a sample. The reagents may include multiple sets of isotopic reagents for labelling multiple samples, buffers and isotopic solvents for conducting labelling reactions. The software may contain algorithms for detecting multiplets of peaks corresponding to groups of sample peaks from many samples. Algorithms are also included for converting the intensity of the spectra to relative intensity of sample components. Instructions may be included to follow the methods of quantitative analysis described herein, and may include instructions for extraction of proteins from cells, reaction of proteins and methods for sample clean-up.

### EXAMPLES

The invention is further described by the following examples.

### Example 1

### Quantitative Analysis of Protein Samples Differentially Labelled by Isotopic Chemical Derivatization

### Materials

Formaldehydes, protein standards and other reagents except where noted were obtained from Sigma-Aldrich Chemical Co. (Mississauga, ON). Deuterium oxide (99.9%) was obtained from Cambridge Isotope Laboratories (Woburn, Mass.). All solutions were prepared in deionized double distilled water. Bulk reversed phase column packing material (Poros 50 R1) was obtained from PerSeptive Biosystems.

### Methods

i) Protein labelling: Protein standards, bovine serum albumin, carbonic anhydrase and ovalbumin, were dissolved in water or 0.1% acetic acid (when necessary) to an initial concentration of 100 pmol/μL. Appropriate aliquots of each solution were combined and the sample was lyophilized. The samples were then treated with equal volumes of freshly prepared 1M formaldehyde or isotopic formaldehyde and 1M sodium cyanoborohydride or isotopic cyanoborohydride in 50 mM NaH₂PO₄ (pH 8.5), the exact isotopic variant of the formaldehyde and cyanoborohyride depends on the mass of the desired label, in either H₂O or D₂O, to provide at least 100 fold excess of each reagent. Samples were permitted to react for 18 hours at room temperature with periodic mixing. Labelled protein samples were then stored at 4° C. until cleanup.

The reason that deuterated water was used is that the reducing agent, NaCNBD₃, can under certain conditions, undergo hydrogen exchange with water to form NaCNBH₃. This reducing reagent would not produce the desired isotopically labelled tag. Therefore, the use of deuterated water (or another deuterated solvent) is an important reaction condition when using deuterated reducing agents capable of such exchange.

ii) Sample Mixing: Prior to mixing of the isotopically labelled samples, sufficient 2M ammonium bicarbonate was added to each sample to react with any excess formaldehyde. This step is not absolutely required but it is recommended since excess reagent can react with amine containing buffers, such as TRIS, used in subsequent sample processing steps. After 30 minutes, the appropriate volume of each sample was combined in a preparation.

iii) Gel Electrophoresis: Optionally, the preparation containing the combined samples was subjected to SDS-PAGE using standard conditions which included a 4% stacking gel and a 12% resolving gel in Tris-Glycine buffer systems. After electrophoresis, proteins were visualized by staining methods using either silver nitrate or Coomassie Blue™. Protein bands were excised and subjected to in-gel cysteine carbamidomethylation with DTT/iodoacetamide using routine procedures.

iv) Sample Cleanup: Optionally, when samples were not subjected to SDS-PAGE, the buffer was exchanged before enzymatic digestion of the proteins. This was accomplished using reverse phase (C₄) columns that were laboratory-prepared in gel-loading pipet tips according to routine procedures. Samples were passed three times through columns that had been previously conditioned with methanol followed by 0.1% trifluoroacetic acid (TFA) in water. The column was then washed with 50 μL of aqueous 0.1% TFA and the proteins were eluted with 85% acetonitrile, 0.5% formic acid (2×50 μL) and lyophilized.

v) Enzymatic Digestion: Proteins larger than approximately 5 kDa could be hydrolyzed before being examined by MS. This is usually done using an enzyme, however, one could also use chemical methods for digestion using, e.g. CNBr or acid (HCl, formic acid). In this example, sequencing-grade chymotrypsin (TLCK-treated) was dissolved in 1 mM HCl at 5 ng/μL and diluted 400 fold in 0.1M ammonium bicarbonate. Lyophilized gel bands were covered with the chymotrypsin solution, typically 25-40 μL. Reverse phase cleanup samples were dissolved in 25 μL of the enzyme solution. Digestions were conducted for 24 hours at 37° C. After digestion, gel bands were extracted with 50% acetonitrile containing 0.5% formic acid, according to established procedures. Extracts were lyophilized and stored at 4° C. prior to analysis by MS.

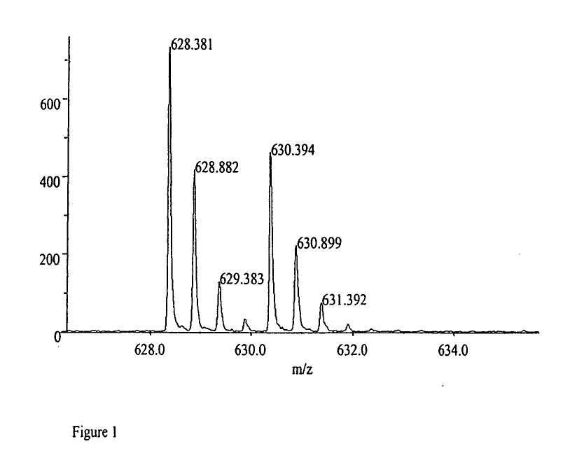

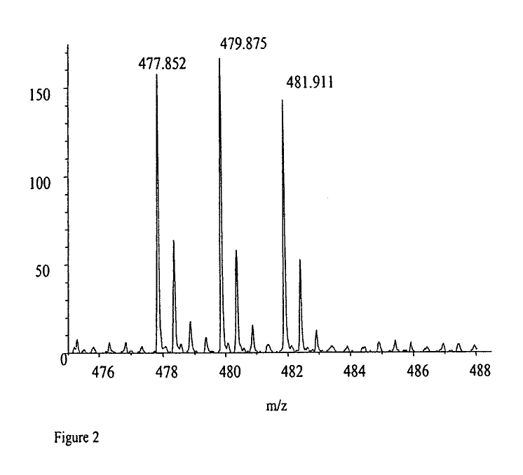

The results of the protein analysis can be found in FIGS. 1 and 2.

FIG. 1 is a MS spectrum of the same protein from two samples, both samples were labelled with formaldehyde, the first sample was reduced with sodium cyanoborohydride and the other sample was reduced with sodium cyanoborodeuteride, the samples were combined and digested with chymotrypsin and analysed using ESI-MS. There are two doubly-charged peptides. In this example, the monoisotopic masses of the [M+H]⁺ of the derivatized peptides are 1255.794 and 1259.780.

FIG. 2 is a MS spectrum as in FIG. 1, except that three samples were labelled as follows. The aldehyde/reducing agent combinations used were: sample 1 formaldehyde/sodium cyanoborohydride; sample 2 formaldehyde/sodium cyanoborodeuteride; sample 3 deuterated formaldehyde/sodium cyanoborohydride. In addition the peptides were triply charged. The spacing of the tags is small enough to minimize the difference in chromatographic properties, but large enough to allow accurate quantitation of even small changes in protein expression. It would not be possible to quantify such small differences in protein levels using conventional staining techniques, such as silver staining.

### Example 2

### Analysis of Doubly Labelled in-Gel Digest of BSA

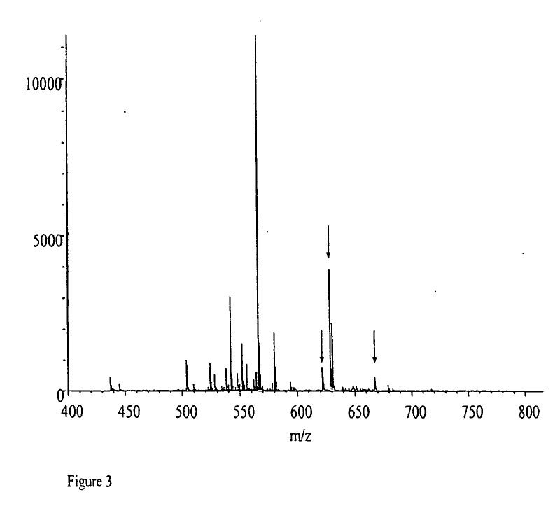

Following the method of Example 1, both BSA samples were labelled with formaldehyde, the first sample was reduced with sodium cayanoborohydride and the other sample was reduced with sodium cyanoborodeuteride, the samples were combined and analysed by SDS-PAGE, excised, digested with trypsin and analysed by LC-MS. FIG. 3 is a LC-MS spectrum demonstrating that the tags were compatible with gel electrophoresis and also demonstrating the added specificity obtained through labelling. Any peak that appeared as a doublet was from the sample and was analysed. The singlet peaks were due to trypsin autolysis products, keratin contamination or sample peptides lacking lysine.

### Example 3

### Analysis of Isotopically Labelled Peptides by Tandem MS

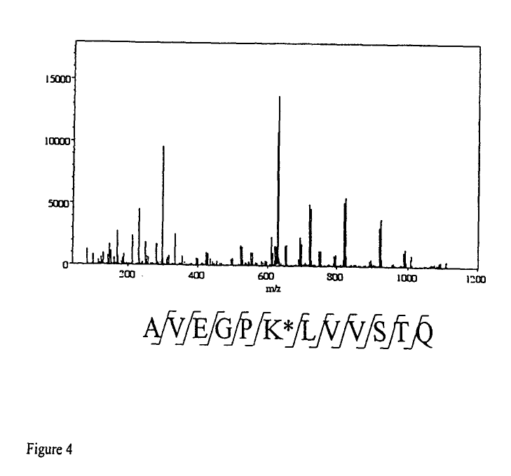

Following the method of Example 1, two BSA samples were isotopically labelled first with formaldehyde, then with sodium cyanoborohydride (sample 1) or sodium cyanoborodeuteride (sample 2), the samples were combined, digested with trypsin, and analysed by tandem MS spectrum. FIG. 4 shows the tandem MS spectrum. Both labelled peptides were fragmented at the same time. This resulted in the appearance of doublet peaks for fragments containing the label (fragments b₆-b₁₁ and y₁-y₇). The tag did not interfere with the ability to perform sequencing experiments through collision-induced fragmentation (CID). The CID process followed a similar pattern to that observed with non-labelled peptides.

### Example 4

### Analysis of Isotopically Labelled Peptides by HPLC-MS

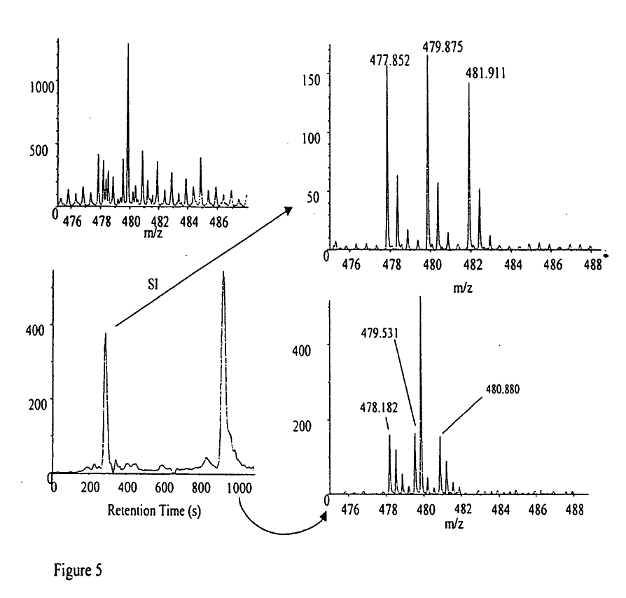

Following the method of Example 1, peptides were isotopically labelled as follows. The aldehyde/reducing agent combinations used were: sample 1 formaldehyde/sodium cyanoborohydride; sample 2 formaldehyde/sodium cyanoborodeuteride; sample 3 deuterated formaldehyde/sodium cyanoborohydride. The samples were combined, digested with trypsin and analysed by HPLC-MS. FIG. 5 is a composite of 4 spectrums that show the added complexity of resolution of peaks by HPLC-MS. Top left, nanoelectrospray spectrum; bottom left, total-ion chromatogram for the m/z range of 475-485; top right, MS spectrum for the first peak consisting of a triplet of doubly charged peptides; bottom right, MS spectrum for a second peak consisting of a triplet to triply charged peptides.

### Example 5

### Analysis of Two Samples of Differentially Labelled BSA

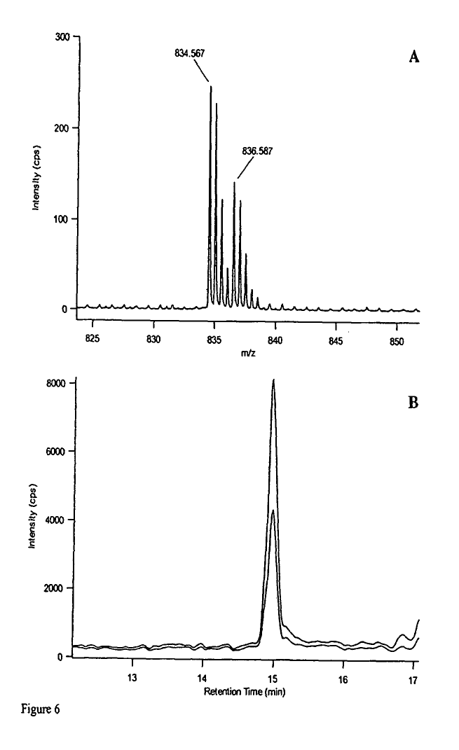

Two samples of BSA were differentially labelled first with formaldehyde, then with sodium cyanoborohydride (sample 1) or sodium cyanoborodeuteride (sample 2). The samples were combined, digested using trypsin and analysed by LC-MS. FIG. 6 shows the resulting MS spectrum and ion chromatogram. Trace A shows the MS spectrum of a pair of labelled tryptic peptides and Trace B shows the extracted ion chromatograms for the same two peptides. The data demonstrates that the labelling did not affect the ability to use trypsin for digestion of the labelled proteins and, furthermore, that the digestion produced doubly charged peptides in the mass range that is suitable for low-energy CID (1000-3000 Da) and subsequent database searching. The data also demonstrates that the labelled peptides eluted at the same time and were chromatographically indistinguishable using standard HPLC conditions. The fact that the labelled peptides co-eluted is ideal for accurate quantitation.

### Example 6

### Analysis of Membrane Proteins From Two Samples of Aeromonas salmonicida Grown Under Different Conditions

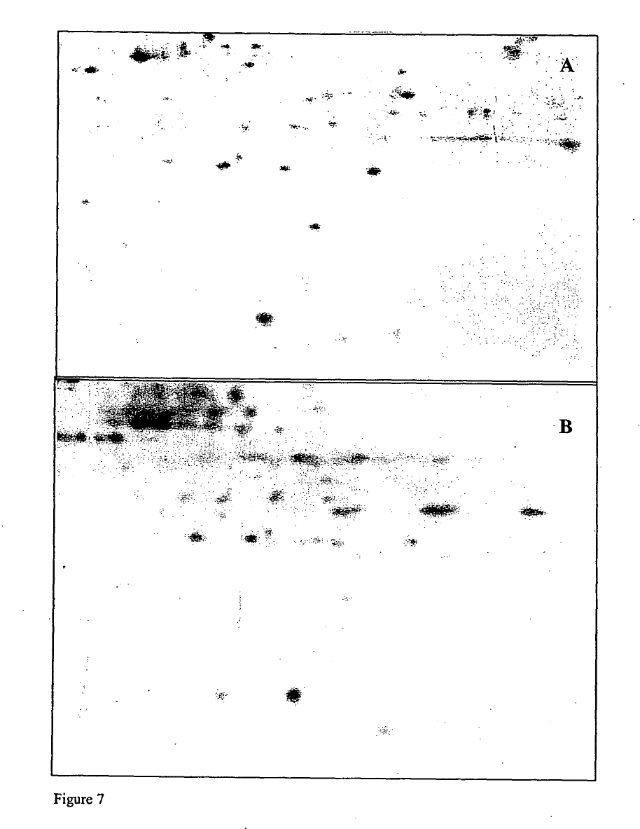

Two samples of Aeromonas Salmonicida were grown under different conditions. The first sample was grown using standard cell-culture conditions and the second sample was grown as a biofilm in minimal media. The two samples were differentially labelled. The first sample was labelled with formaldehyde and sodium cyanoborohydride and the second sample was labelled with formaldehyde and sodium cyanoborodeuteride. The samples were combined and digested with trypsin and then separated by 2D Gel electrophoresis as described in Example 1 and shown in FIG. 7. Panel A is a separation of membrane proteins from one sample of the bacteria Aeromonas Salmonicida. Panel B is a separation of the two samples of Aeromonas Salmonicida grown under different conditions and differentially labelled. The proteins were extracted from cultured bacteria and the separation was performed using isoelectric focusing (pH 3-11) for approximately 12 hours followed by SDS-PAGE on a 12% acrylamide gel. The gels were stained with silver. This example demonstrates the compatibility of the differential labelling technology for processing multiple samples in the same gel.

### Example 7

### Analysis of Various Differentially Labelled Amines

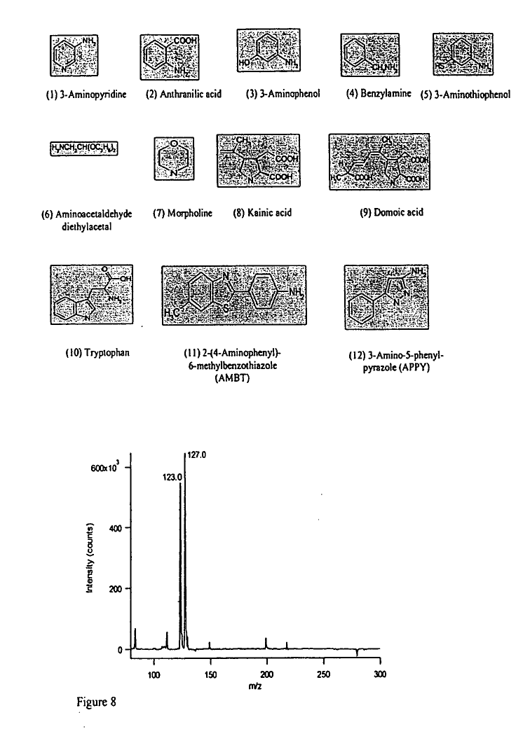

Various amines were labelled with either CH₂O or CD₂O and reduced with sodium cyanoborohydride or sodium cyanoborodeuteride in acetonitrile which contained 10% (v/v) acetic acid or acetic acid d₄. Various amounts of each sample were mixed and analysed by LC-MS. No digestion was required, as the molecules were small. The MS used was a triple-quadrupole instrument (API III+) with an IonSpray source operated in the positive-ion mode. Table 2 provides the details of the analysis. Column 1 lists the names of the amines. Column 2 lists the mass of the protonated pseudo-molecular ion. Columns 3 and 4 list the amount of differentially labelled amine combined for the analysis. Columns 5 and 6 list the expected and experimentally determined ratios, respectively. Finally, column 7 lists the calculated percent error. FIG. 8 shows the molecular structure of the amines. The lower panel shows the mass spectrum of 3-aminothiophenol labelled with CH₂O (m/z=123.0) and CD₂O (m/z=127.0) and NaCNBH₃. The expected ratio of intensities was 1.07 and the observed ratio was 1.10, corresponding to an error of 2.7%.

TABLE 2
Analysis of various amines

| | | mass of CH₂O | mass of CD₂O | | | |
|---|---|---|---|---|---|---|
| | [M + H] + | labelled cmpd | labelled cmpd | Expected | Observed | |
| Compound | (Da) | (mg) | (mg) | Ratio | Ratio | % error |
| 3-Aminopyridine | 95.1 | 52.5 | 56.1 | 0.94 | 0.88 | −5.8 |
| Aminoacetaldehyde | 134.2 | 59.3 | 54.9 | 1.08 | 0.98 | −9.3 |
| diethylacetal | | | | | | |
| 3-Aminophenol | 110.1 | 52.1 | 48.0 | 1.09 | 1.01 | −6.8 |
| Anthranilic acid | 138.2 | 49.4 | 58.9 | 0.84 | 0.77 | −8.7 |
| 3-Aminothiophenol | 126.2 | 51.3 | 48.0 | 1.07 | 1.10 | 2.7 |
| Benzylamine | 108.2 | 42.9 | 55.3 | 0.78 | 0.66 | −14.8 |
| Morpholine | 88.1 | 57.0 | 57.2 | 1.00 | 0.98 | −1.9 |
| Tryptophan | 205.2 | 42.8 | 50.9 | 0.84 | 1.00 | 18.7 |
| Kainic acid | 214.2 | 39.6 | 44.4 | 0.89 | 0.95 | 6.9 |
| | | 40.3 | 13.2 | 3.05 | 2.95 | −3.2 |
| | | 11.5 | 56.1 | 0.20 | 0.24 | 16.6 |
| Domoic acid | 312.2 | 39.6 | 44.4 | 0.89 | 1.05 | 17.6 |
| | | 40.3 | 13.2 | 3.05 | 3.34 | 9.5 |
| 3-Amino-5-phenyl- | 160.2 | 32.3 | 68.6 | 0.47 | 0.51 | 7.4 |
| Pyrazole (APPY) | | | | | | |
| 2-(4-Aminophenyl)- | 241.2 | 28.6 | 60.0 | 0.48 | 0.42 | −12.9 |
| 6-methylbenzothiazole | | | | | | |
| (AMBT) | | | | | | |

The data demonstrates that the labelling is compatible with primary amines, including aliphatic compounds (1), aromatic compounds (2)-(5), (10)-(12), and secondary amines (7)-(9). Further, the aromatic heterocyclic nitrogen atoms are not methylated whereas the aliphatic nitrogens are labelled. The compounds in (8) and (9) are representative of food toxins, and the compounds of (10)-(12) are representative of drugs.

### Example 8

### Analysis of Four Samples of Differentially Labelled Amine Containing Molecules

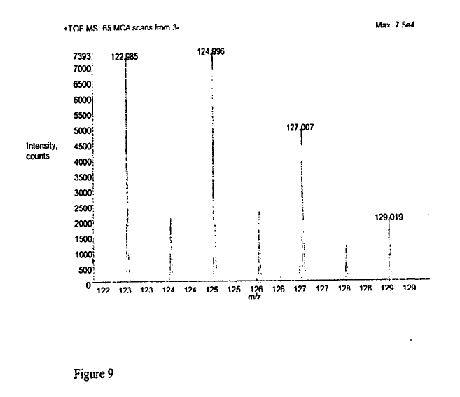

In this example, four samples of 3-aminopyridine were labelled by the method described in Example 7. Specifically, sample 1 formaldehyde/sodium cyanoborohydride; sample 2 formaldehyde and sodium cyanoborodeuteride; sample 3 deuterated formaldehyde/sodium cyanoborohydride; sample 4 deuterated formaldehyde/sodium cyanoborodeuteride. The samples were analyzed by MALDI-MS. The data are displayed in FIG. 9. The peaks at m/z 122.98, 124.99. 127.01 and 129.02 correspond to the labelled amines from the various samples. The expected ratio was 1:1.06:0.43:0.2 and the ratio obtained was 1:1.03:0.62:0.25.

### Example 9

### Analysis of a Preparation Containing Five Different Concentrations of Ovalbumin, Each Differentially Labelled

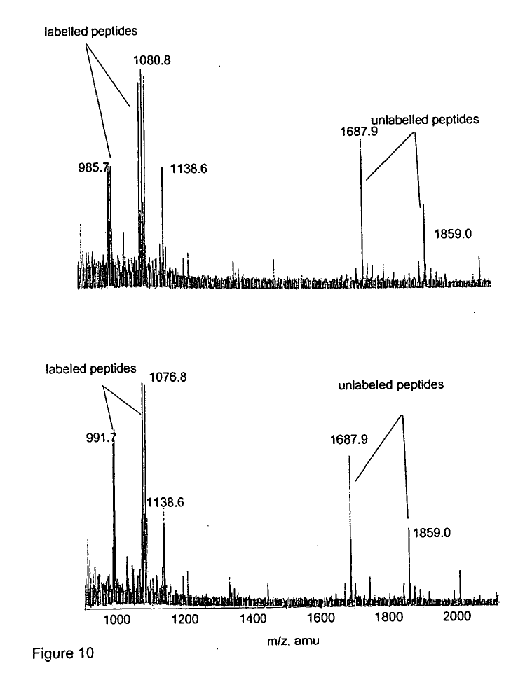

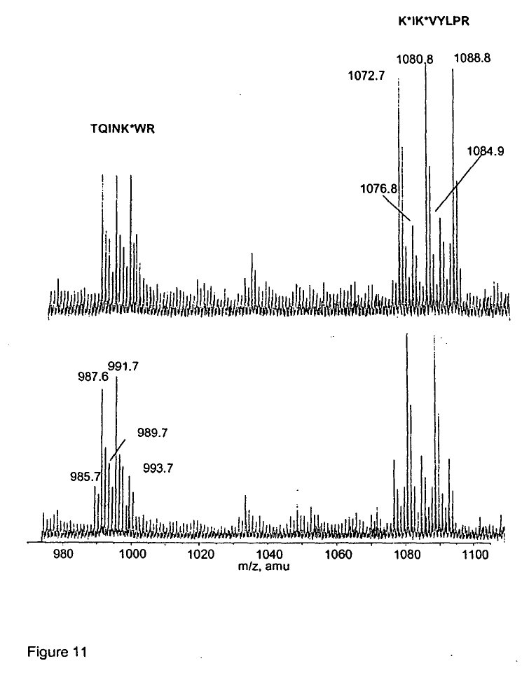

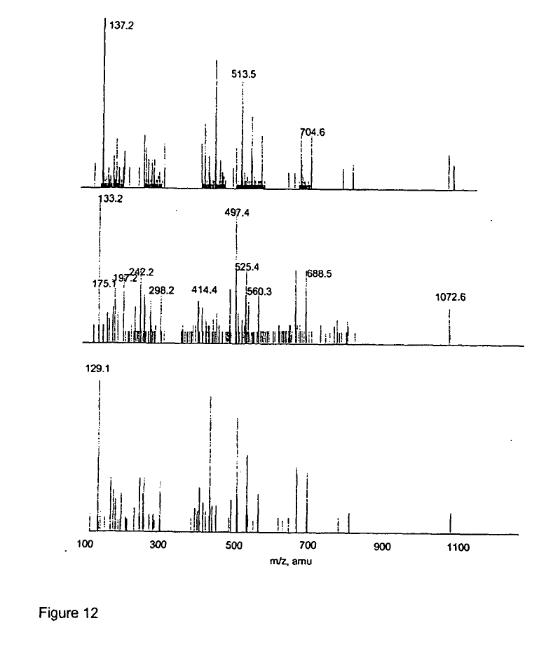

Each sample was treated with formaldehyde or isotopic formaldehyde and NaCNBH₃/H₂O or NaCNBD₃/D₂O. Samples were combined in a preparation and proteins separated by SDS gel electrophoresis. Gels were visualized using silver staining, bands or spots excised and digested with trypsin. Tryptic digests were analyzed by MALDI QqTOF-MS. 500 fmol of protein was loaded on the MALDI target. FIG. 10 shows the mass spectra of the samples. In the top trace, the protein ratios were 3:1:3:1:3 and in the bottom trace the protein ratios were 1:3:1:3:1. A mixture of labelled and unlabelled peaks are present, both of which can be used for identification. FIG. 11, is an expansion of FIG. 10 in the region of the labelled peptides. FIG. 12 shows the Tandem MS spectra of peaks with m/z ratios of 1072.8 (middle trace), 1080.8 (bottom trace) and 1088.8 (top trace). The spectra were acquired using MALDI-QqTOF-MS/MS. The fragmentation pattern is identical for all of the labels. In addition, the low-mass ions at 129 (CH₃ label), 133 (¹³CH₂D label)and 137 (¹³CD₃ label) are indicative of the specific label present.

### Example 10

### Comparison of the Proteins Formed in Candida albicans at 30° C. versus 37° C.

Proteins isolated from Candida albicans grown at two different conditions were isolated. The fungus was grown under conditions favouring the yeast form (30° C.) and conditions favouring the hyphal form (37° C.+serum). Proteins were extracted, isotopically labelled, combined in a preparation, separated by SDS-PAGE electrophoresis, digested with trypsin and analysed by MALDI-QqTOF-MS

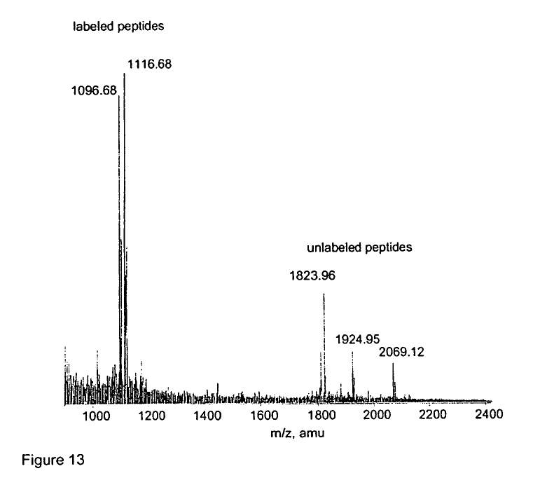

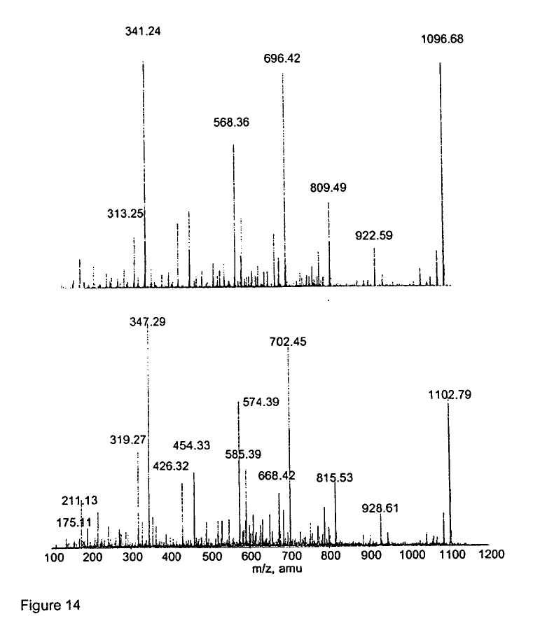

FIG. 13 is the MS spectrum obtained from a labelled protein isolated by SDS-PAGE, excised and digested with trypsin. FIG. 14 contains the Tandem MS spectra of peaks with m/z ratios of 1096.7 (top trace) and 1102.8 (bottom trace) as in FIG. 13. The spectra were acquired individually using MALDI-QqTOF-MS/MS. Note that the fragmentation pattern is identical for both of the labels. Both spectra matched to the protein enolase.

Although the invention has been described with examples, it is to be understood that modifications may be resorted to as will be apparent to those skilled in the art. Such modifications and variations are to be considered within the purview and scope of the present invention.

### References

1. T. P. Conrads, K. Alving, T. D. Veenstra, M. E. Belov, G. A. Anderson, D. J. Anderson, M. S. Lipton, L. Pasa-Tolic, H. R. Udseth, W. B. Chrisler, B. D. Thrall and R. D. Smith. Quantitative analysis of bacterial and mammalian proteomes using a combination of cysteine affinity tags and ¹⁵N metabolic labelling. Anal. Chem., 73 2132-2139 (2001).

2. M. Geng, J. Ji and F. E. Regnier. Signature-peptide approach to detecting proteins in complex mixtures. J. Chromatography, A. 870, 295-313 (2000).

3. D. R. Goodlett, A. Keller, J. D. Watts, R. Newitt, E. C. Yi, S. Purvine, J. Eng, P. van haller, R. Aebersold and E. Kolker. Differential stable isotope labelling of peptides and de novo sequence derivation. Rapid Commun. Mass Spectrom., 15, 1214-1221 (2001).

4. M. B. Goshe, T. P. Conrads, E. A. Panisko, N. H. Angell, T. D. Veenstra and R. D. Smith. Phosphoprotein isotope-coded affinity tag approach for isolating and quantitating phosphopeptides in proteome-wide analyses. Anal. Chem., 73, 2578-2586 (2001).

5. S. P. Gygi, B. Rist, S. A. Gerber, F. Turecek, M. H. Gelb and R. Aebersold. Quantitative analysis of complex protein mixtures using isotope-coded affinity tags. Nature Biotech. 17, 994-999 (1999).

6. J. Ji, A. Chakraborty, M. Geng, X. Zhang, A. Amini, M. Minou and F. Regnier. Strategy for qualitative and quantitative analysis in proteomics based on signature peptides. J. Chromatography, B, 745, 197-210 (2000).

7. O. A. Mirgorodskaya, Y. P. Kozmin, M. I. Titov, R. Korner, C. P Sonksen and P. Roepstorff. Quantitation of peptides and proteins by matrix assisted laser desorption/ionization mass spectrometry using ¹⁸O-labelled internal standards. Rapid Commun. Mass Spectrom., 14, 1226-1232 (2000).

8. M. Molloy and P. C. Andrews. Phosphopeptide derivatization signatures to identify serine and threonine phosphorylated peptides by mass spectrometry. Anal. Chem, ASAP article (2001).

9. M. Munchbach, M. Quadroni, G. Miotto and P. James. Quantitation and facilitated de novo sequencing of proteins by isotopic N-terminal labelling of peptides with a fragmentation-directing moiety. Anal. Chem., 72, 4047-4057 (2001).

10. Y. Oda, K. Huang, F. R. Cross, D. Cowbum and B. T. Chait. Accurate quantitation of protein expression and site-specific phosphorylation., Proc. Natl. Acad. Sci. 96, 6591-6596 (1999).

11. R. D. Smith, L. Pasa-Tolic, M. S. Lipton, P. K. Jensen, G. A. Anderson, Y. Shen, T. P. Conrads, H. R. Udseth, R. Harkewicz, M. E. Belov, C. Masselon and T. D. Veenstra. Rapid quantitative measurements of proteomes by Fourier transform ion cyclotron resonance mass spectrometry. Electrophoresis, 22, 1652-1668 (2001).

12. S. Wang and F. Regnier. Proteomics based on selecting and quantifying cysteine containing peptides by covalent chromatography. J. Chromatography, A, 924, 345-357 (2001).

13. W. Weckwerth, L. Willmitzer and O. Fiehn. Comparative quantification and identification of phosphoproteins using stable isotope labelling and liquid chromatography/mass spectrometry. Rapid Commun. Mass Spectrom. 14, 1677-1681 (2000).

14. X. Yao, A. Freas, J. Ramirez, P. A. Demirev and C. Fenselau. Proteolytic ¹⁸O labelling for comparative proteomics: model studies with two serotypes of adenovirus. Anal. Chem., 73, 2836-2842 (2001).

What is claimed is:

1. A method for the simultaneous quantitative analysis of at least three samples of molecules, comprising:
(i) reacting the molecules of each sample with a set of at least two isotopically labelled reagents, wherein each set of isotopically labelled reagents is differentially labelled, resulting in at least three differentially and isotopically labelled derivatives of molecules;
(ii) combining the derivatized molecules in a preparation for examination by mass spectrometry; and
(iii) examining the preparation by mass spectrometry.

2. A method for the quantitative analysis of a sample of molecules having an amine bearing an active hydrogen, comprising:
(i) reacting the molecules, with isotopically labelled reagents resulting in the reductive alkylation of the amines to their alkylamine derivatives, such that the alkylamine derivatives are isotopically labelled in a preparation for examination by mass spectrometry; and
(ii) examining the preparation by mass spectrometry.

3. A method for the quantitative analysis of two or more samples of molecules having an amine bearing an active hydrogen, comprising:
(i) reacting the molecules in each sample with isotopically labelled reagents resulting in the reductive alkylation of the amines to their alkylamine derivatives, such that the alkylamine derivatives are isotopically labelled;
(ii) combining the derivatized molecules in a preparation for examination by mass spectrometry; and
(iii) examining the preparation by mass spectrometry.

4. The method of claim 1, 2 or 3 comprising an additional step of cleaving the derivatized molecules in the preparation into fragments, prior to examining the preparation by mass spectrometry.

5. The method of claim 1, 2 or 3 comprising an additional step of denaturing the molecules prior to reacting the molecules with isotopically labelled reagents.

6. The method of claim 1, 2, 3 or 4 wherein the step of examining the preparation by mass spectrometry comprises introducing the preparation containing the derivatized molecules or fragments to a mass spectrometer using electrospray ionisation.

7. The method of claim 6 wherein the electrospray ionisation method is selected from a group consisting of nanospray, pneumatically assisted electrospray, ionspray and turboionspray.

8. The method of claim 1, 2 or 3 comprising an additional step of separating the derivatized molecules in the preparation before examining the preparation by mass spectrometry.

9. The method of claim 8 wherein the step of separating the derivatized molecules uses a separator selected from a group consisting of 1-D gel electrophoresis, SDS-PAGE, isoelectric focusing, 2-D gel electrophoresis, zone electrophoresis, isotachophoresis, ion exchange chromatography, normal phase chromatography, reverse phase chromatography, hydrophobic interaction chromatography, size exclusion chromatography and any combination of these separators.

10. The method of claim 4 comprising an additional step of separating the fragments after cleaving the derivatized molecules in the preparation.

11. The method of claim 10 wherein the step of separating the fragments uses a separator selected from a group consisting of liquid chromatography, high performance liquid chromatography and capillary electrophoresis.

12. The method of claims 1, 2 or 3 comprising an additional step of analyzing the preparation after examining the preparation by mass spectrometry.

13. The method of claim 12 wherein the step of analyzing the preparation is selected from a group consisting of collision-induced dissociation in a mass spectrometer operating in MS/MS mode, peptide mass fingerprinting, peptide mapping, Edman sequencing and sequencing by sequential amino acid cleavage.

14. The method of claim 13, comprising an additional step, after the step of analyzing the preparation of sequencing the molecule.

15. The method of claim 1, 2 or 3 wherein the isotopically labelled reagents are an aldehyde and a reducing agent.

16. The method of claim 15 wherein the aldebyde is selected from a group consisting of formaldehyde and acetaldehyde.

17. The method of claim 15 wherein the reducing agent is selected from a group consisting of a sodium cyanoborohydride, sodium borohydride, dialkyl borane complexes and pyridine borane complexes.

18. The method of claim 1, 2 or 3 wherein the sample is selected from a group consisting of cells, cellular extracts, sub-cellular extracts, cellular lysates, peptides, proteins, drugs, toxins, antibodies and pollutants.

19. The method of claim 18 wherein the proteins are extracted from cells.

20. The method of claim 19 wherein the amines of the proteins are selected from a group consisting of lysine residues, omithine residues and residues at the N-terminal amino group of the proteins.

21. The method of claim 1, 2 or 3 wherein the step of examining the preparation by mass spectrometry utilizes a mass spectrometer selected from a group consisting of:
(i) Fourier transform—Ion cyclotron resonance mass spectrometers (FT-ICR-MS);
(ii) Time of Flight mass spectrometers (TOF-MS, TOF-TOF-MS);
(iii) Ion trap mass spectrometers (IT);
(iv) Quadrupole mass spectrometers (Q-MS and QqQ-MS);
(v) Ion mobility mass spectrometers (IM-MS);
(vi) Quadrupole (or hexapole, octapole)-Time of Flight mass spectrometers (Q-TOF, and Qq-TOF); and
(vii) Ion trap—Time of flight mass spectrometers (IT-TOF).

22. The method of claim 21 comprising an additional step of combining the mass spectrometer with an ionisation source.

23. The method of claim 22 wherein the ionisation source is selected from a group consisting of electrospray ionisation, matrix-assisted laser desorption and ionisation (MALDI), field desorption, thermal desorption and laser desorption.

24. A preparation of three samples of molecules for simultaneous quantitative analysis by mass spectrometry, each sample comprising differentially and isotopically labelled derivatives of molecules, each sample resulting from a reaction of a set of at least two isotopically labelled reagents with the molecules.

25. A preparation of a sample of molecules comprising isotopically labelled derivatives resulting from the reductive alkylation of the amines to alkylamine derivatives by isotopically labelled reagents.

26. A preparation of two or more samples of molecules for the simultaneous analysis by mass spectrometry, each sample comprising differentially and isotopically labelled derivatives of molecules resulting from the reductive alkylation of the amines to alkylamine derivatives by isotopically labelled reagents.

27. Use of a mass spectrometer for the analysis of a sample according to any of claims 1, 2 or 3.

28. A kit comprising isotopically labelled reagents and instructions to follow the methods of quantitative analysis of any of claims 1, 2 or 3.

29. A method for the quantitative analysis of two or more cellular extracts comprising molecules having an amine bearing an active hydrogen, comprising:
(i) reacting the molecules of the extracts with isotopically labelled reagents resulting in the reductive alkylation of the amines to their alkylamine derivatives, such that the alkylamine derivatives are isotopically labelled;
(ii) combining the derivatized molecules of the extracts in a preparation;
(iii) separating the molecules;
(iv) enzymatically cleaving the molecules into fragments;
(v) separating the fragments;
(vi) examining the fragments by mass spectrometry; and
(vii) sequencing the fragments.
